(19) United States
(12) Patent Application Publication
(10) Pub. No.: US 2024/0172922 A1
(43) Pub. Date: May 30, 2024

(54) DISTENDABLE BEACON DEVICES, SYSTEMS, AND METHODS

(71) Applicant: BOSTON SCIENTIFIC SCIMED, INC., MAPLE GROVE, MN (US)

(72) Inventors: Jeff Gray, Sudbury, MA (US); Katharine Eckerline, Plymouth, MN (US); Kyle P. Moore, Hopkinton, MA (US); Natalie Byrom, Boston, MA (US)

(73) Assignee: BOSTON SCIENTIFIC SCIMED, INC., MAPLE GROVE, MN (US)

(21) Appl. No.: 18/522,585

(22) Filed: Nov. 29, 2023

Related U.S. Application Data
(60) Provisional application No. 63/428,799, filed on Nov. 30, 2022.

(51) Int. Cl. A61B 1/00

(52) U.S. Cl. CPC A61B 1/00085; A61B 1/00042; A61B 1/00148

(57) ABSTRACT

A locator device and system with an expandable element facilitating imaging of the locator device at a target site at a first anatomical location within a patient from a remote second anatomical location within the patient. The locator device may be formed of a material capable of being imaged from the remote second anatomical location. The locator device may be expandable to contact the interior of the anatomical structure at the first anatomical location to facilitate deployment and to resist movement with respect thereto. The locator device may be separable from the delivery system used to deliver the locator device and left in place at the target site. The locator device may expand the anatomical structure at the first anatomical location to facilitate imaging of such structure from the remote second anatomical structure. In some instances, the locator device may be returned to the delivery configuration and removed from the target site.

### CROSS-REFERENCE TO RELATED APPLICATIONS

This application claims the benefit of priority under 35 U.S.C. § 119 to U.S. Provisional Application No. 63/428,799, filed Nov. 30, 2022, the entire disclosure of which is hereby incorporated by reference herein for all purposes.

### FIELD

The present disclosure relates generally to devices, systems, and methods used in performing procedures within a body, such as endoscopic procedures. More particularly, the present disclosure relates generally to devices, systems, and methods used to locate an anatomical site within a patient's body.

### BACKGROUND

Viewing, locating, and manipulating anatomies, devices, and/or anatomies containing one or more devices from within a body may be difficult. During a procedure within the body, such as an endoscopic procedure within a body lumen (not involving cutting open the body), a medical professional may need to externally locate a particular anatomical structure of and/or position within the body. Locating a desired anatomical structure of and/or position within a body may be difficult due to a lack of or low amount of illumination, and/or intervening anatomy, and/or the shape and/or configuration of various portions of the body. It is with respect to these and other considerations that the present improvements may be useful.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a locator device, such as for locating or identifying an anatomical site within a patient, has an expandable member having a distal end and a proximal end and shiftable between a delivery configuration and an expanded deployed configuration; a capsule having a distal end and a proximal end, the proximal end of the expandable member being coupled to the distal end of the capsule; and a securement feature positioned to hold the expandable member in the expanded deployed configuration. The locator device is deliverable to a target site within a patient in the delivery configuration and imageable from a different site within the patient when in the expanded deployed configuration.

In some aspects, the locator device is deliverable to the target site with a delivery system and separable from the delivery system to be deployed at the target site.

Optionally, the locator device further includes an actuator extending proximally from the distal end of the expandable member and retractable proximally to shift the expandable member from the delivery configuration to the expanded deployed configuration. Optionally, the actuator includes a pull wire extending proximally to a proximal end controllable by a medical professional to shift the expandable member between the delivery configuration and the expanded deployed configuration. Optionally, the pull wire has a proximal section extending to the proximal end thereof, and a distal section extending from the proximal section to the distal end of the expandable member; and the proximal pull wire section is separable from the distal pull wire section to deploy the locator device at the target site. Optionally, the actuator includes one or more actuator elements each extending from a distal end at the distal end of the expandable member to a proximal end within the capsule. Optionally, the securement feature is coupled adjacent the proximal end of at least one of the one or more actuator elements to engage with the capsule. Optionally, the proximal end of at least one of the one or more actuator elements is biased outwardly to secure the securement feature within a window defined in the capsule upon proximal retraction of the at least one of the one or more actuator elements to shift the expandable member to the expanded deployed configuration. Optionally, the securement feature is releasable from engagement within the window to allow the expandable member to return to the delivery configuration. Optionally, the expandable member is formed of a shape memory material configured to return the expandable member to the delivery configuration.

In some embodiments, the expandable member is formed of a plurality of elongated elements formed of a shape memory material configured to bow outwardly into an expanded basket-type configuration upon application of force thereto, and configured to return the expandable member to the delivery configuration upon release of the force.

In accordance with various principles of the present disclosure, a locator system, such as for locating or identifying an anatomical site within a patient, includes a locator device comprising an expandable member shiftable between a delivery configuration and an expanded deployed configuration sized to engage the interior of the anatomical structure in which the locator device is deployed; and a delivery system operatively associated with the locator device. The locator device is separable from the delivery system to be deployed at the target site in the expanded deployed configuration.

Optionally, the locator system further includes an actuator extending proximally from the distal end of the expandable member and retractable proximally to shift the expandable member from the delivery configuration to the expanded deployed configuration. Optionally, the actuator includes a pull wire extending proximally to a handle at a proximal end of the delivery system controllable by a medical professional to shift the expandable member between the delivery configuration and the expanded deployed configuration; and the pull wire is separable from the expandable member to allow separation of the locator device from the delivery system.

Optionally, the locator device further includes a capsule having a distal end and a proximal end, the proximal end of the expandable member being coupled to the distal end of the capsule; and a securement feature positioned to hold the expandable member in the expanded deployed configuration.

In accordance with various principles of the present disclosure, a method of locating a target site at a first anatomical location from a site at a second anatomical location includes delivering a locator device to the target site, the locator device being configured to shift between a delivery configuration and an expanded deployed configuration; shifting the locator device to an expanded configuration; and imaging the expanded location device from the second anatomical location.

Optionally, the locator device comprises an expandable member formed of a material imageable from the second anatomical location.

Optionally, the method further includes expanding the anatomical wall at the first anatomical location to facilitate imaging thereof at the second anatomical location.

Optionally, the method of further includes securing the locator device in the expanded configuration and separating the locator device from the delivery system used to deliver the locator device to the target site to deploy the locator device at the target site. Optionally, the method further includes returning the locator device to the delivery configuration and removing the locator device from the target site.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an example of an embodiment of a locator system formed in accordance with various aspects of the present disclosure being navigated through a schematic representation of a gastrointestinal environment.

FIG. 2A illustrates an elevational view of a locator system with a locator device in a delivery configuration.

FIG. 2B illustrates an elevational view similar to that of FIG. 2A, but with the locator device in a deployed configuration.

FIG. 3A illustrates an example of an embodiment of a locator device deployed in a schematic representation of a gastrointestinal deployment site.

FIG. 3B illustrates an end view of the locator device illustrated in FIG. 3A.

FIG. 4A illustrates a stage of a procedure being performed once the position of a locator device such as illustrated in FIG. 3A has been determined.

FIG. 4B illustrates a further stage of a procedure performed once the position of the locator device illustrated in FIG. 4A has been determined.

FIG. 4C illustrates a further stage of the procedure illustrated in FIG. 4B.

FIG. 4D illustrates a further stage of the procedure illustrated in FIG. 4C .FIG. 5A illustrates a cross-sectional view along line VA-VA of a detail of FIG. 2A.

FIG. 5B illustrates a cross-sectional view along line VB-VB of a detail of FIG. 2B.

FIG. 6 illustrates an example of an embodiment of a locator device and an example of an embodiment of returning the locator device to a delivery configuration from a deployed configuration.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, etc.) such location or site.

The present disclosure relates to devices, systems, and methods useful in performing a procedure, such as, without limitation, an endoscopic, laparoscopic, and/or open surgical procedure, within the body by initially identifying the target site for the procedure with a locator system, deploying the locator system at the target site, and then (e.g., at a later time) locating the locator system to perform the procedure at the desired target site identified by the locator system. A number of medical procedures require identification of the location of an anatomical structure, such as an organ or a tissue wall, and then delivering medical instruments to a second, different anatomical location to perform a procedure on or at the first-identified location. The first-identified location may be alternately referenced herein as a “target location”, a “target site”, a “treatment site”, a “deployment site”, a “target tissue”, a “target tissue site”, etc., including combinations thereof and other grammatical forms thereof, without intent to limit. The second anatomical location or site may be referenced interchangeably herein as a different anatomical location, a separate anatomical location, a remote location, an imaging site, a locating site, an access location, etc., including combinations thereof and other grammatical forms thereof, without intent to limit. As may be appreciated, the terms site and location may be used interchangeably herein without intent to limit. Typically, principles of the present disclosure are applied with respect to a target location and a different anatomical location located internally within a patient. For instance, the target location and the imaging location may be separated by a tissue wall, and/or may be in different body organs or cavities. It will be appreciated that reference to “at” the target tissue site is intended to include tissue at and about the vicinity of (e.g., along, adjacent, etc.) the target tissue, and is not limited to just target tissue.

Non-limiting examples of procedures which may be performed with respect to a target location different from an imaging location include various medical procedures which involve moving a tissue wall (e.g., a body lumen wall or the wall of an organ) to a desired position, such as relative to another tissue wall (e.g., a body lumen wall or the wall of an organ). For instance, various procedures may be performed by entering the gastrointestinal (GI) tract through a first organ or structure (such as the esophagus, stomach, duodenum, small intestine, large intestine, or peritoneal cavity), and delivering an anchor or stent to adjacent organs or lumen or tissue structures (such as an adjacent portion of the GI tract, the bile duct, the pancreatic duct, the gallbladder, the pancreas, cysts, pseudocysts, abscesses, and the like). Typically, it is necessary to penetrate both an access tissue wall (e.g., a wall of a first organ or a first body lumen) at the access location, through which access is established to the target location, and a second tissue wall (e.g., of a wall of a second organ or a second body lumen) along or adjacent or at the target location at which the procedure is to be performed. A tissue anchor or stent or other device may be deployed between/across adjacent body lumens, organs, or other structures, such as to maintain tissue walls thereof in apposition, and/or to create an anastomosis, as indicated by the procedure. Tissue anchors may be used, in addition to the stent(s), to secure adjacent tissues or organs, such as before a stent is deployed, and may be left in place after the stent has been deployed.

Endoscopic procedures may be preferred over open surgical (involving cutting open the patient's body to gain access into the patient's body and internal anatomical structures) or laparoscopic (typically involving smaller incisions to gain access into the patient's body and internal anatomical structures) procedures for various reasons. For instance, open surgical or even laparoscopic procedures may involve longer recovery times than endoscopic methods which do not involve incisions, but which, instead, typically access the internal anatomy through a natural orifice and advancing equipment, tools, devices, etc., transluminally to the target area. However, endoscopic procedures present challenges with regard to locating a target location from a different anatomical location within the patient's body. In accordance with various principles of the present disclosure, a locator device and/or associated locator system is deployed at the target location to identify the target location from the different anatomical location.

An example of a common endoscopic procedure presenting challenges in identifying a target location is a gastric bypass procedure in which a connection, such as an anastomosis, is created between the stomach and a certain part of the intestines through a gastroenteral anastomosis. For example, conditions of a disease may be benefitted by bypassing a portion of the duodenum, such as by creating an anastomosis, e.g., about 150 cm or greater from the pylorus, between the stomach and the small intestines (with a distal portion of the duodenum, or with the jejunum). In gastric outlet obstruction, a gastrojejunostomy serves the purpose of draining the contents of the stomach into the jejunum below/distal to the obstructed and/or dysfunctional duodenum. A gastrojejunostomy procedure may also serve as a minimally invasive and possibly reversible treatment option for patients with metabolic disease, by creating an anastomosis between the stomach and the jejunum to bypass the duodenum, with accompanying desired metabolic effects. In this manner, absorption of stomach contents (e.g., food and other nutrients) in the duodenum may be bypassed, and nutrients from such contents may not be absorbed, or uptake or absorption may be delayed, as such contents travel from the stomach through the small bowel, promoting patient weight loss and possibly controlling or resolving type-2 diabetes.

Endoscopic procedures, such as gastroenteral anastomoses, present various challenges, including the need to locate, endoscopically, a desired anatomical position (e.g., in the intestines) through a tissue wall, such as in a different anatomical location (e.g., via the gastric lumen). Ultrasound and/or fluoroscopy procedures provide images through anatomical walls (e.g., the gastric and enteral walls). Fluoroscopy is commonly used in endoscopy suites as it is well suited for viewing dense materials, such as those from which medical instruments are made. The location of a target site within the body may make identification of such location challenging. For instance, because of the sheer length, extent, and windings of the small intestines, locating a target site along the small intestines presents challenges for various imaging techniques and systems, and to medical professionals in general.

The use of a locator system at the target location in accordance with various principles of the present disclosure may facilitate identification of the target location from a different anatomical location within the patient's body. The locator device is inserted, such as with an endoscope, to the desired location for the procedure (e.g., where the connection of the intestines with the stomach is intended to be made). The endoscope is withdrawn, leaving the locator device in place. Then, the endoscope is advanced to the imaging site (optionally after being removed from the patient and the locator system, and then being re-inserted). One or more instruments for performing the procedure are inserted into and through the endoscope, such as to make an incision at the imaging site to access the target location (e.g., through the stomach wall and into the peritoneum). It will be appreciated that terms such as instruments, tools, devices, etc., may be used interchangeably herein without intent to limit. The locator device is visualized, or otherwise located, such as with the use of the endoscope, to identify the target site for the procedure, and the procedure may thus be performed at the desired target site. For instance, once in the stomach, the physician locates the desired section of the small intestine with the aid of a locator device within the small intestine (such as may be seen from outside the intestinal wall). The outside of the small intestine is accessed (e.g., by cutting through the stomach wall and extending a grasper through the peritoneum) and grasped and brought together with the stomach. A stent may then be deployed to connect the small intestine and the stomach, thereby creating the anastomosis.

In accordance with various principles of the present disclosure, a locator system includes a delivery device and a locator device. In accordance with various principles of the present disclosure, the locator device is configured to shift between a delivery configuration and an expanded deployed configuration. The locator device may alternately be referenced herein as an expander for the sake of convenience and without intent to limit. In some embodiments, the locator device has a locator member which shifts between a delivery configuration and an expanded deployed configuration. In some embodiments, the locator member, which may alternately be referenced herein as an expandable member for the sake of convenience and without intent to limit, may be held in the expanded deployed configuration. Additionally or alternatively, the locator device may be separated from the delivery device used to deliver the locator device to the target site, such as with the locator member in the expanded deployed configuration. The expanded target site and/or the locator member may be imaged by any of a variety of imaging systems or techniques to locate the target site from another location within the patient's body.

The locator member is formed from a material capable of being imaged by any of a variety of imaging systems positioned at a location different from the target site. In some embodiments, the locator device is capable of being shifted from the deployed configuration back to the delivery configuration for withdrawal from the deployment site. In some embodiments, an additional locator element is provided in connection with the locator device, such as a signal generator (e.g., light emitting diode).

Various embodiments of locator devices, systems, and methods will now be described with reference to examples illustrated in the accompanying drawings. Reference in this specification to “one embodiment,” “an embodiment,” “some embodiments”, “other embodiments”, etc., indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc., in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. The following description is of illustrative examples of embodiments only, and is not intended as limiting the broader aspects of the present disclosure.

It will be appreciated that common features are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). Moreover, certain features in one embodiment may be used across different embodiments and are not necessarily individually labeled when appearing in different embodiments.

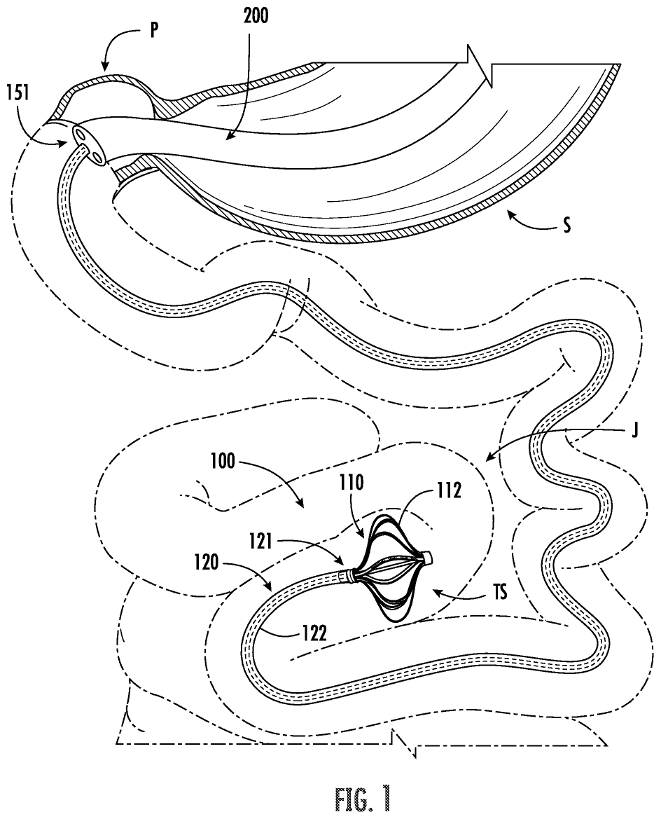

Turning now to the drawings, an example of an embodiment of an environment in which a locator system 100 formed in accordance with various principles of the present disclosure may be used is illustrated in FIG. 1. The locator system 100 includes a locator device 110 delivered with the assistance of a delivery system 120 (operatively associated with the locator device 110) to a desired target site TS. The delivery system 120 includes a flexible elongate element 122 facilitating delivery of the locator device 110, such as through tortuous passages within the patient's body. In the example illustrated in FIG. 1, the locator device 110 is delivered to a portion of a patient's jejunum J to be brought into apposition with a portion of the patient's stomach S, as described in further detail below. As may be appreciated, the flexible elongate element 122 must navigate through a tortuous pathway through various turns of the jejunum J. An endoscope 200 may be used to deliver the locator system 100, but typically does not extend significantly past the duodenum into the small intestines.

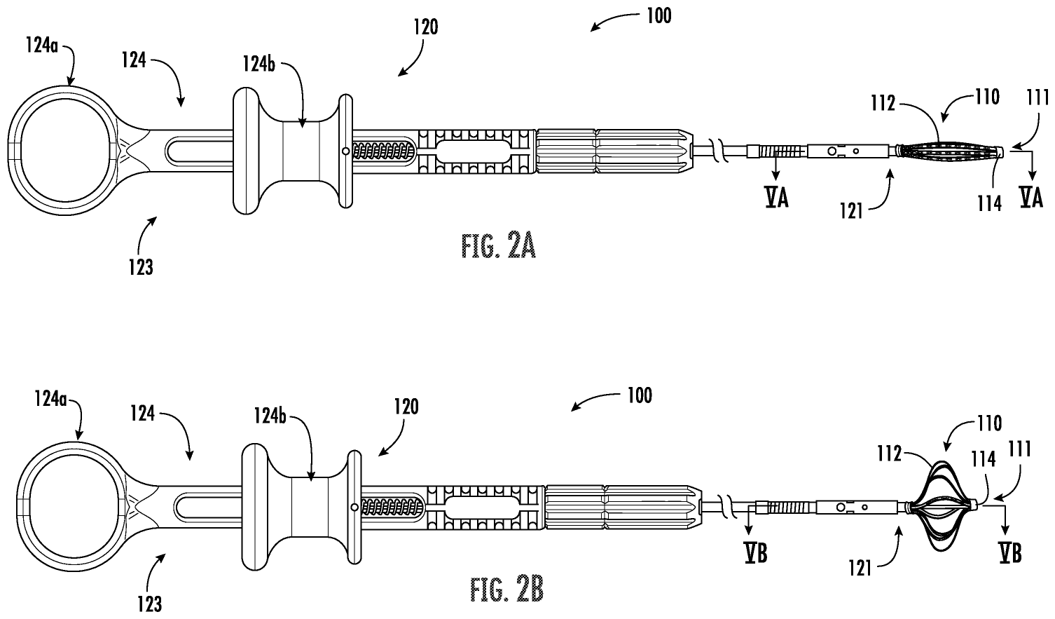

In accordance with various principles of the present disclosure, the locator device 110 is capable of being shifted between a delivery configuration, such as illustrated in FIG. 2A, and an expanded configuration, such as illustrated in FIG. 2B. The locator device 110 may thus alternately be referenced herein as an expander 110. The delivery system 120 may include a handle 12 (e.g., at a proximal end 123 of the delivery system 120) operatively associated with the locator device 110 to shift the locator device 110 between the delivery and expanded configurations, as will be described in further detail below. For instance the handle 124 may include a proximal section 124a (e.g., a thumb ring) and a distal section 124b (e.g., a finger grasping section or “half spool”) moved with respect to each other to actuate the locator device 110.

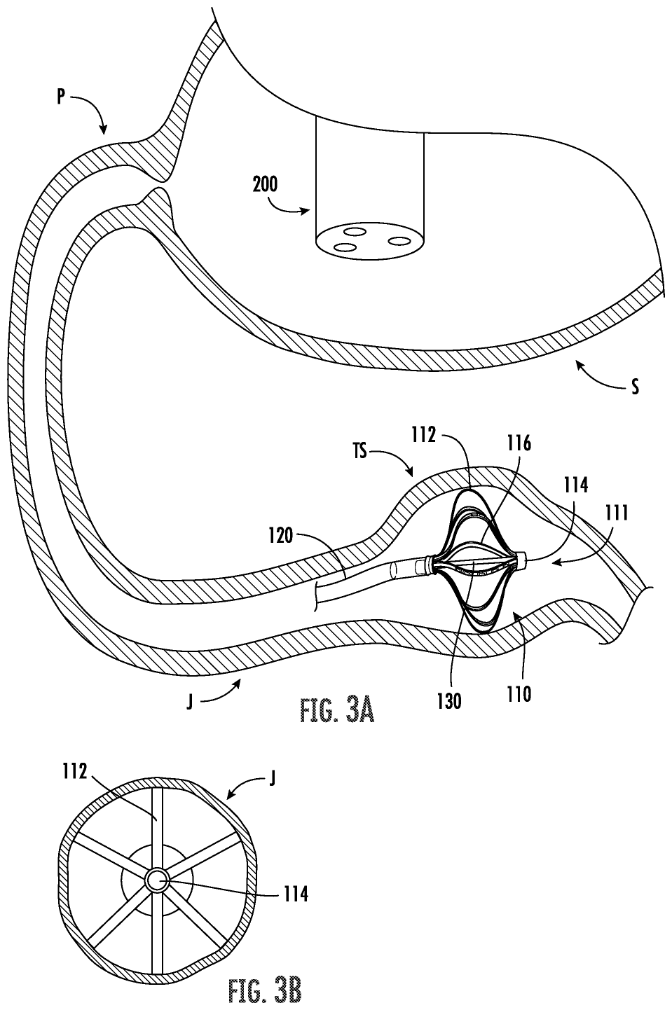

When in the expanded configuration, the locator device 110 expands in diameter to engage the interior of a body passage or lumen. In the illustrated example of an embodiment, the locator device 110 foreshortens as it expands in diameter. The locator device 110 may expand sufficiently to contact the interior (inner wall) of the anatomical structure (e.g., body passage or lumen) in which the locator device 110 is positioned, such as to facilitate deployment and to resist movement with respect thereto. In some embodiments, the locator device 110 expands sufficiently to expand the diameter of the body passage/lumen. In some embodiments, the locator device 110 is held or maintained in its expanded configuration to hold open a body passage or lumen (such terms being used interchangeably herein without intent to limit), such as illustrated in FIG. 3A. Expansion of the size/dimensions of the body lumen, such as with the assistance of the locator device 110, makes the target site TS easier to identify/locate from a remote location, as may be appreciated by those of ordinary skill in the art. The locator device 110 may be formed of an imageable material and/or may include markers capable of being imaged from a remote location. Examples of such materials include a biocompatible metal, such as Nitinol, stainless steel; or an imageable biocompatible polymer such as nylon, an imide, acetal, etc., such as may be imageable with the use of fluoroscopy. Expansion of the size/dimensions of the locator device 110 facilitates imaging thereof from a remote location, as may be appreciated by those of ordinary skill in the art. Moreover, expansion of the size dimensions of the anatomical structure in which the locator device 110 is positioned also facilitates imaging and identification of the target site TS.

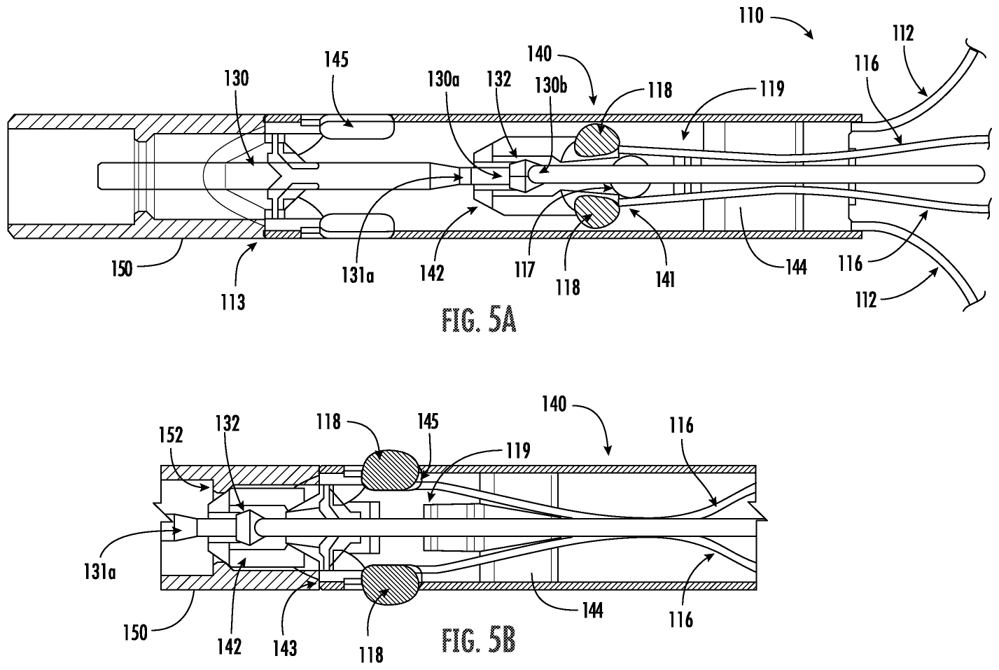

In some embodiments, the locator device 110 is formed of one or more expansion elements 112. The examples of embodiments of expansion elements 112 in FIG. 3A are illustrated as flexible/bendable beams, although other configurations are within the scope and spirit of the present disclosure, as may be appreciated by those of ordinary skill in the art. At least three expansion elements 112 may be provided to hold open a body lumen (e.g., a portion of the small intestines), such as illustrated in the end view of FIG. 3B. The expansion elements 112 may form a “basket” type arrangement. An actuator is provided to shift the locator device 110 between the delivery configuration and the expanded configuration. A handle 124, such as described above, may be used to shift the actuator to shift the locator device 110. In the illustrated example of an embodiment, a pull wire 130 is provided as an actuator, and extends along the expansion elements 112 (e.g., through a “basket” type arrangement of expansion elements 112) to be coupled with a distal end 111 of the locator device 110, and optionally with one or more of the expansion elements 112. The distal ends 115 of the expansion elements 112 and the distal end 131 of the pull wire 130 may be coupled in any of a variety of manners known to those of ordinary skill in the art (e.g., welding, adhesive bonding, mechanical deformation, etc.). In the example of an embodiment illustrated in FIG. 3A, the distal ends 115 of the expansion elements 112 and the distal end 131 of the pull wire 130 are joined along and/or by an end cap 114. The end cap 114 may be made of a radiopaque element or otherwise configured to be imageable by available imaging systems and techniques. Proximal retraction (e.g., pulling) on the pull wire 130 (e.g., via the handle 124) proximally retracts the distal end thereof and the distal ends of the expansion elements 112 (and the end cap 114, if provided), causing outward flexing of the expansion elements 112 to expand the locator device 110. Additional or alternative actuators may be provided, in addition to the pull wire 130, to cause expansion of the locator device 110, such as described in further detail below with reference to FIG. 5A and FIG. 5B.

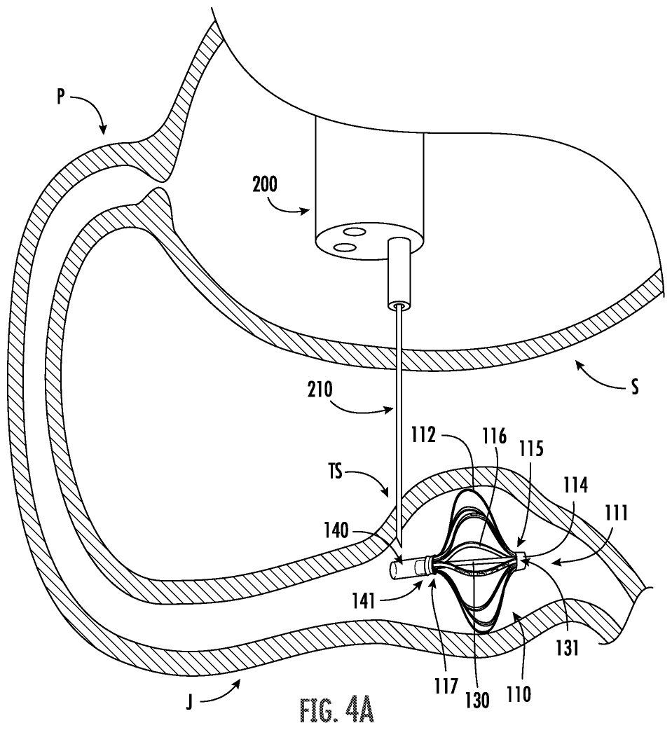

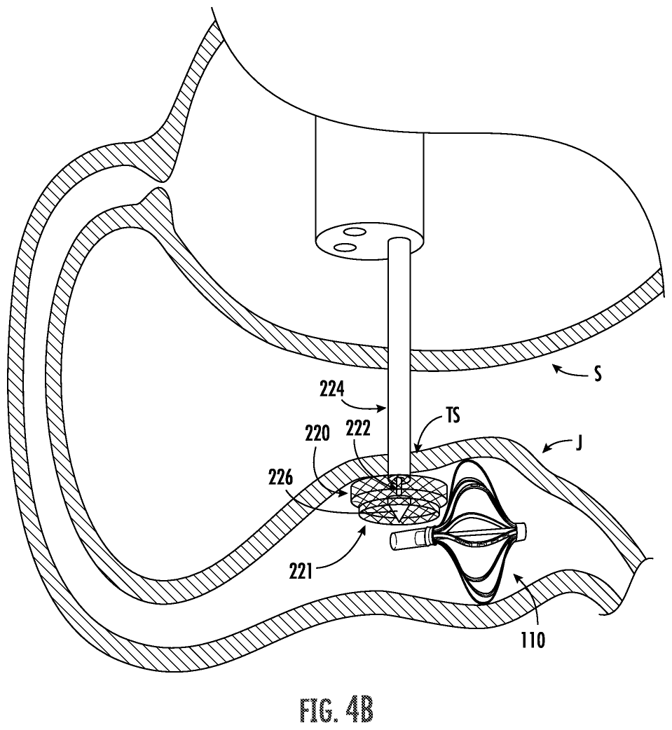

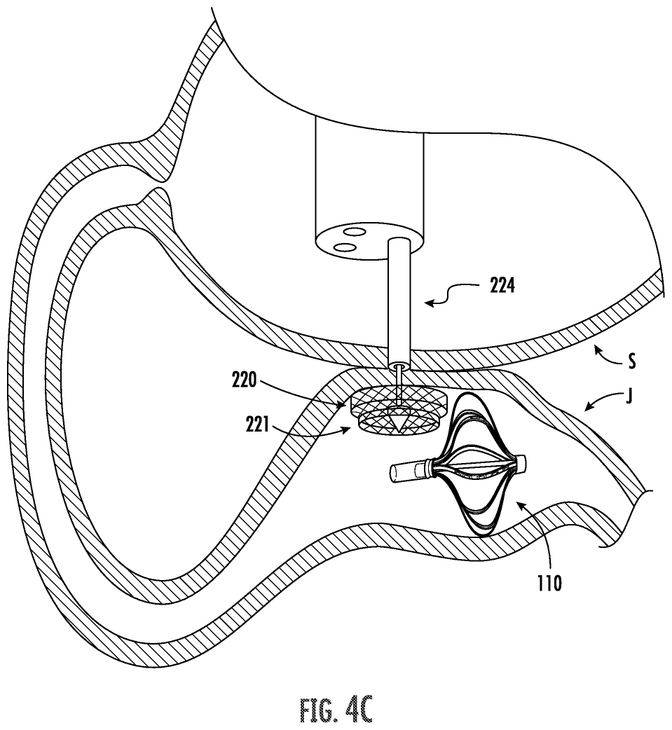

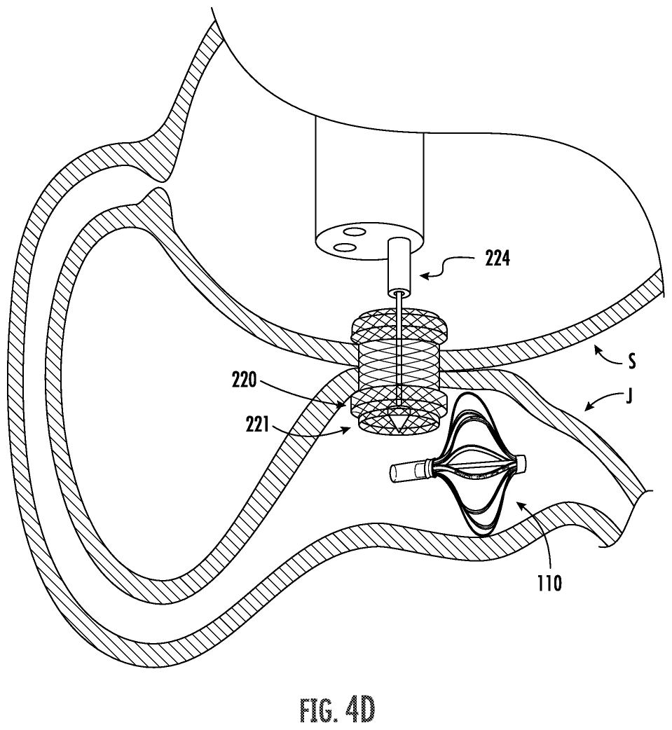

In some aspects, the locator system 100 delivers the locator device 110 with the use of an endoscope 200, such as illustrated in FIG. 1. The endoscope 200 may also be used at a separate anatomical location to locate the target site TS from a position at the separate anatomical location. For instance, as illustrated in FIG. 4A, the endoscope 200 may be withdrawn from the locator device 110 at the target site TS and repositioned at another site, such as another anatomical site within the patient. As illustrated in FIG. 4A, with the locator device 110 expanded at a target site TS in the jejunum J, the endoscope 200 may be positioned in (e.g., relocate to) the stomach S to locate the target site TS through the use of an imaging device or system or technique capable of imaging the locator device 110 and/or indicator thereon and/or expanded target site TS, such as described above, the present disclosure not being limited in this regard.

It may be desirable to deploy the locator device 110 at the target site TS and to withdraw the delivery system 120 therefrom to allow ready repositioning of the endoscope 200 from a delivery location to a separate location at which the locator device 110 is to be identified/located. For instance, in the example of an embodiment illustrated in FIG. 1 and the example of an embodiment illustrated in FIG. 3A, the endoscope 200 is moved from the pylorus P to the stomach S. The example of an embodiment of a locator device 110 illustrated in FIG. 4A is separable from the delivery system 120, such as in a deployed, expanded configuration. Once the locator device 110 has been deployed, a procedure may be performed with respect to the target site TS, as illustrated in FIGS. 4A-4D. As illustrated in FIG. 4A, an endoscope 200 (such as the endoscope 200 which delivered the delivery system 120 and the locator device 110, repositioned in the stomach S) delivers instruments, such as a needle 210, for performing a procedure with respect to the target site TS. It will be appreciated that the locator device 110 may be delivered distal to the target site TS to facilitate performance of a procedure at the target site TS (such as illustrated in FIG. 4A), or proximal to the target site TS, such as depending on the procedure and/or the preference of the medical professional performing the procedure. The needle 210 is illustrated as extending through the wall of the stomach S and through the wall of the jejunum J to the target site TS, such as to facilitate delivery of further instruments to the target site TS. To perform a gastrojejunostomy, a distal end 221 of a stent 220 is delivered into the jejunum J at the target site TS, as illustrated in FIG. 4B. The stent 220 may be delivered over a delivery shaft 222, and within a delivery sheath 224 (e.g., in a manner described in U.S. Patent Application Publication 2020/0330156, titled Methods And Devices For Access Across Adjacent Tissue Layers, published on Oct. 22, 2022, and incorporated herein in its entirety for all purposes). The delivery shaft 222 may have a penetrating end 226 configured to facilitate enlarging openings through the walls of the stomach S and the jejunum J to allow passage of the delivery sheath 224 and the stent 220 therethrough. The jejunum J is drawn proximally towards the stomach S and the delivery sheath 224 is proximally withdrawn, as illustrated in FIG. 4C and in FIG. 4D to extend the stent 220 across the walls of the jejunum J and the stomach S and to allow the distal end 221 and the proximal end 223 of the stent 220 to seat against the walls of the jejunum J and the stomach S, respectively, to hold such tissue walls in apposition (or at least closer together), as illustrated in FIG. 4D. Optionally, the illustrated locator device 110 is also retrievable, and, may be shiftable from the expanded configuration back to the delivery configuration for transluminal retrieval from the patient (e.g., through an endoscope 200), as discussed in further detail below.

In accordance with various principles of the present disclosure, the locator device 110 may be shifted between a delivery configuration and an expanded configuration in any of a variety of manners. Moreover, the locator device 110 may be coupled with a delivery system 120 (such as illustrated in FIG. 2A and FIG. 2B), such as along a distal end 121 thereof, and separable therefrom in any of a variety of manners. An example of an embodiment of elements and mechanisms allowing a locator device 110 such as illustrated FIG. 2A and FIG. 2B and in FIG. 4A to shift between a delivery configuration and an expanded configuration and optionally to be separated from a delivery system 120 (such as illustrated in FIG. 2A and FIG. 2B) is illustrated in FIG. 5A and FIG. 5B.

In the example of an embodiment illustrated in FIG. 5A, the pull wire 130 is illustrated extending proximally through a capsule 140 at a proximal end 113 of the locator device 110. The pull wire 130 may extend proximally along or through a lumen within the flexible elongate element 122. The proximal end of the pull wire 130 (not shown) extends proximally to be controlled by a medical professional (e.g., pulled to cause the locator device 110 to expand). Optionally, the pull wire 130 is coupled to the handle 124 of the delivery system 120. The handle 124 of the delivery system 120 may include a proximal grip element 124a and a distal grip element 124b, such as illustrated in and noted with reference to FIG. 2A and FIG. 2B. The grip elements 124a, 124b of the handle 124 may be movable with respect to each other such as to actuate the locator device 110 (e.g., to expand the locator device 110). For instance, the pull wire 130 may be coupled to the proximal grip element 124a to be movable therewith in any manner acceptable to one of ordinary skill in the art. Movement of the proximal grip element 124a thus causes proximal movement/retraction of the pull wire 130. As noted above with reference to FIG. 3A, the pull wire 130 extends distally to the distal end 111 of the locator device 110. Proximal pulling on the pull wire 130 (such as via the handle 124) retracts the distal end 111 of the locator device 110 towards the capsule 140. Proximal ends 117 of the expansion elements 112 may be coupled to the distal end 141 of the capsule 140, as illustrated in FIG. 4A and FIG. 5A, in any of a variety of manners known to those of ordinary skill in the art (e.g., welding, brazing, soldering, adhering with an appropriate adhesive, mechanical interference, etc.). As the pull wire 130 is retracted proximally, pulling the distal end 111 of the locator device 110 proximally, the distal ends 115 of the expansion elements 112 are pulled proximally towards the distal end 141 of the capsule 140 and the expansion elements 112 flex/bend/bow (such terms being used interchangeably herein without intent to limit) outwardly to shift the locator device 110 into an into an expanded configuration such as illustrated in FIG. 2B and FIG. 4A.

In some embodiments, the locator device 110 includes one or more actuator elements 116 which may extend along the expansion elements 112 when the locator device 110 is in the delivery configuration (e.g., illustrated in FIG. 2A). In some embodiments, proximal ends 119 of the actuator elements 116 are operatively associated with (e.g., coupled to or mounted with respect to) a yoke 142 movably positioned within the capsule 140. The yoke 142 is coupled with the pull wire 130. Proximal movement of the pull wire 130 thus not only proximally retracts the distal end 111 of the locator device 110, but also proximally retracts the yoke 142 and the proximal ends 119 of the actuator elements 116. The actuator elements 116 thus facilitate foreshortening of the locator device 110 to shift into a radially expanded configuration, and may be considered additional or auxiliary actuators of the locator device 110. The actuator elements 116 may simply be mounted over the yoke 142 to move therewith, or may be coupled with the yoke 142. In some embodiments, the actuator elements 116 are operatively associated with a guide 144. The guide 144 may be coupled with the yoke 142 to move therewith so that proximal pulling on the pull wire 130 proximally retracts the yoke 142 and the guide 144. The yoke 142 and guide 144 may be separably coupled to facilitate deployment of the locator device 110 and separation from the delivery system 120, such as described in further detail below.

In some embodiments, securement features 118 are provided along proximal ends 119 of actuator elements 116 of the locator device 110 to hold the locator device 110 (e.g., the expansion elements 112 thereof) in an expanded configuration. As the yoke 142 is proximally retracted within the capsule 140, the securement features 118 are proximally retracted as well. The yoke 142 is proximally retracted until the securement features 118 reach respective windows 145 in the capsule 140, at which point the securement features 118 extend radially outwardly into the respective windows 145, such as illustrated in FIG. 5B. Engagement of the securement features 118 within the windows 145 of the capsule 140 hold the actuator elements 116 in place with respect to the capsule 140 and thus maintain the locator device 110 in the expanded configuration.

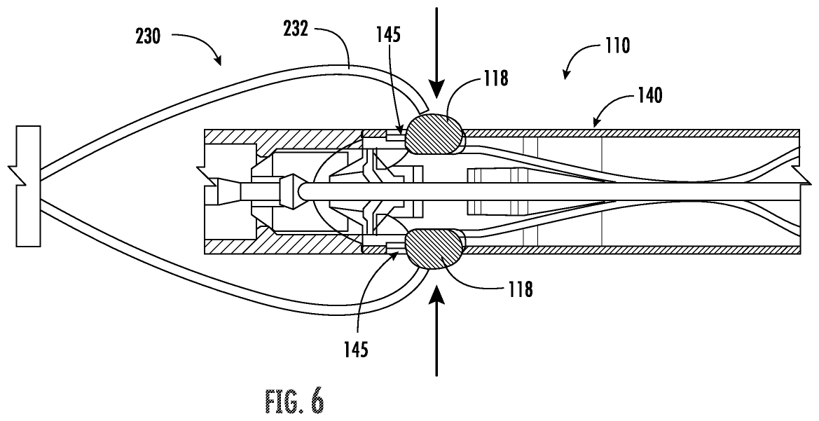

To return the locator device 110 to a delivery configuration (such as illustrated in FIG. 2A) from an expanded configuration (such as illustrated in FIG. 2B or FIG. 4A), an end effector 230 (such as a grasper or forceps with movable jaws 232) may be inserted into the patient and advanced to the locator device 110, such as illustrated in FIG. 6 (without illustrating an example of an environment of the locator device 110). The jaws 232 may push the securement features 118 back into the capsule 140 (e.g., radially inwardly into the respective windows 145). The expansion elements 112 may be formed of a shape memory material configured to return to a generally elongated/straight configuration to return the locator device 110 to a delivery configuration once the securement features 118 are no longer held in place with respect to the capsule 140. The force of such shape memory material may pull the distal end 111 of the locator device 110 distally away from the capsule 140, pulling the proximal ends 119 of the actuator elements 116, and the securement features 118, away from the windows 145.

In some aspects, it may be desirable to deploy the locator device 110, leaving the locator device 110 at the target site TS. In such embodiment, various features and elements may be provided to facilitate detachment/separation of the locator device 110 from the delivery system 120. For instance, in some embodiments, the pull wire 130 is separated from the locator device 110 so that the pull wire 130 does not extend proximally from the locator device 110 once the locator device 110 has been deployed. In the example of an embodiment illustrated in FIG. 5A, the pull wire 130 is formed with a proximal pull wire section 130a and a distal pull wire section 130b which are separable from each other. The proximal pull wire section 130a and the distal pull wire section 130b may both be coupled to the yoke 142 and/or to each other (e.g., within the yoke 142). However, the proximal pull wire section 130a is separably coupled with respect to the locator device 110 whereas the distal pull wire section 130b preferably does not separate from the locator device 110. The proximal pull wire section 130a may be separable from the locator device 110 in any of a variety of manners as may be appreciated by those of ordinary skill in the art. For instance, the distal end 131a of the proximal pull wire section 130a is separably coupled with the yoke 142 or may be frangible, such as to break to be separated from the locator device 110. In the example of an embodiment illustrated in FIG. 5A and FIG. 5B, the proximal pull wire section 130a has an expanded end 132 (e.g., in the form of a spherical element such as a ball, or any other configuration wider than the remaining diameter of the pull wire 130) held in place within the yoke 142. Upon a threshold amount of pulling force, the expanded end 132 may be releasable from its seat within the yoke 142 or the proximal pull wire section 130a may be frangible proximally adjacent to the expanded end 132 to separate the proximal pull wire section 130a from the expanded end 132. In some embodiments, the yoke 142 may be pulled and retracted proximally until the yoke 142 extends within and/or engages a bushing 150 coupled to a proximal end 143 of the capsule 140. The bushing 150 is configured to prevent the yoke 142 from being withdrawn from the capsule 140. For instance, a narrowed section 152 may be formed within the bushing 150 to form a limit stop or hard stop for the capsule 140. Once proximal withdrawal of the capsule 140 is stopped by the bushing 150, such as by the narrowed section 152 therein, continued pulling on the pull wire 130 causes the proximal pull wire section 130a to separate from the expanded end 132 thereof.

In embodiments in which the yoke 142 is proximally retracted beyond the point at which the securement features 118 extend into the windows 145 of the capsule 140, the actuator elements 116 separate from the yoke 142 to allow the yoke 142 to continue to be proximally retracted. In some embodiments, the actuator elements 116 are simply mounted over the yoke 142 in a manner allowing the yoke 142 to proximally retract the actuator elements 116 (to expand the locator device 110). Once the securement features 118 extend into the windows 145, the securement features 118 disengage from the yoke 142, allowing the yoke 142 to continue proximal movement with respect to the capsule 140. For instance, the yoke 142 may be shaped to engage the proximal ends 119 of the actuator elements 116 when the securement features 118 are contained within the capsule 140. However, once the securement features 118 extend radially outwardly into the windows 145 of the capsule 140, the proximal ends 119 of the actuator elements 116 disengage the yoke 142 and allow separate movement of the yoke 142 with respect to the actuator elements 116. If the actuator elements 116 are operatively associated with a guide 144, then the guide 144 may be separably coupled with the yoke 142 in any of a variety of manners known to those of ordinary skill in the art to allow the yoke 142 to continue to move proximally after the securement features 118 have been engaged within the windows 145 in the yoke 142 without the guide 144 interfering with the securement features 118.

The locator device 110 may thus be deployed and separated from the delivery system 120 and the endoscope 200 may be withdrawn readily therefrom and moved to another location (e.g., to the patient's stomach S, as illustrated in FIG. 3A) from which the expanded locator device 110 and expanded target site TS may be identified/located. It will be appreciated that if the locator device 110 is separated from the delivery system 120, the endoscope 200 need not be removed from the patient's body to be repositioned to another location, as may be necessary if the pull wire 130 remains coupled with the locator device 110 and extends outside the patient's body to actuate the locator device 110.

It will be appreciated that all apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure.

Although embodiments of the present disclosure may be described with specific reference identifying a target site in the gastrointestinal system, it should be appreciated that the disclosed medical devices, systems, and methods may be used to identify target sites at other locations within a patient's body, such as the abdominal cavity, digestive system, urinary tract, reproductive tract, respiratory system, cardiovascular system, circulatory system, and the like.

Various further benefits of the various aspects, features, components, and structures of a locator device and system such as described above, in addition to those discussed above, may be appreciated by those of ordinary skill in the art.

All of the devices and/or methods disclosed and claimed herein can be made and executed without undue experimentation in light of the present disclosure. While the devices and methods of this disclosure have been described in terms of selected embodiments, it may be apparent to those of skill in the art that variations can be applied to the devices and/or methods and in the steps or in the sequence of steps of the method described herein without departing from the concept, spirit, and scope of the disclosure. All such similar substitutes and modifications apparent to those skilled in the art are deemed to be within the spirit, scope, and concept of the disclosure as defined by the appended claims.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, and joined) are to be construed broadly and may include intermediate members between a collection of elements and relative movement between elements unless otherwise indicated. As such, connection references do not necessarily infer that two elements are directly connected and in fixed relation to cach other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A locator device comprising:
an expandable member having a distal end and a proximal end and shiftable between a delivery configuration and an expanded deployed configuration;
a capsule having a distal end and a proximal end, the proximal end of said expandable member being coupled to the distal end of said capsule; and
a securement feature positioned to hold said expandable member in the expanded deployed configuration;
wherein said locator device is deliverable to a target site within a patient in the delivery configuration and imageable from a different site within the patient when in the expanded deployed configuration.

2. The locator device of claim 1, wherein said locator device is deliverable to the target site with a delivery system and separable from the delivery system to be deployed at the target site.

3. The locator device of claim 1, further comprising an actuator extending proximally from the distal end of said expandable member and retractable proximally to shift said expandable member from the delivery configuration to the expanded deployed configuration.

4. The locator device of claim 3, wherein said actuator includes a pull wire extending proximally to a proximal end controllable by a medical professional to shift said expandable member between the delivery configuration and the expanded deployed configuration.

5. The locator device of claim 4, wherein:
said pull wire has a proximal section extending to the proximal end thereof, and a distal section extending from said proximal section to the distal end of said expandable member; and
said proximal pull wire section is separable from said distal pull wire section to deploy said locator device at the target site.

6. The locator device of claim 3, wherein said actuator includes one or more actuator elements each extending from a distal end at the distal end of said expandable member to a proximal end within said capsule.

7. The locator device of claim 6, wherein said securement feature is coupled adjacent the proximal end of at least one of said one or more actuator elements to engage with said capsule.

8. The locator device of claim 7, wherein the proximal end of at least one of said one or more actuator elements is biased outwardly to secure said securement feature within a window defined in said capsule upon proximal retraction of said at least one of said one or more actuator elements to shift said expandable member to the expanded deployed configuration.

9. The locator device of claim 8, wherein said securement feature is releasable from engagement within the window to allow said expandable member to return to the delivery configuration.

10. The locator device of claim 9, wherein said expandable member is formed of a shape memory material configured to return said expandable member to the delivery configuration.

11. The locator device of claim 1, wherein said expandable member is formed of a plurality of elongated elements formed of a shape memory material configured to bow outwardly into an expanded basket-type configuration upon application of force thereto, and configured to return said expandable member to the delivery configuration upon release of the force.

12. A locator system comprising:
a locator device comprising an expandable member shiftable between a delivery configuration and an expanded deployed configuration sized to engage the interior of the anatomical structure in which the locator device is deployed; and
a delivery system operatively associated with said locator device;
wherein said locator device is separable from the delivery system to be deployed at the target site in the expanded deployed configuration.

13. The locator system of claim 12, further comprising an actuator extending proximally from the distal end of said expandable member and retractable proximally to shift said expandable member from the delivery configuration to the expanded deployed configuration.

14. The locator system of claim 13, wherein:
said actuator includes a pull wire extending proximally to a handle at a proximal end of said delivery system controllable by a medical professional to shift said expandable member between the delivery configuration and the expanded deployed configuration; and
said pull wire is separable from said expandable member to allow separation of said locator device from said delivery system.

15. The locator system of claim 14, wherein said locator device further comprises:
a capsule having a distal end and a proximal end, the proximal end of said expandable member being coupled to the distal end of said capsule; and
a securement feature positioned to hold said expandable member in the expanded deployed configuration.

16. A method of locating a target site at a first anatomical location from a site at a second anatomical location, said method comprising:
delivering a locator device to the target site, the locator device being configured to shift between a delivery configuration and an expanded deployed configuration;
shifting the locator device to an expanded configuration; and
imaging the expanded location device from the second anatomical location.

17. The method of claim 16, wherein the locator device comprises an expandable member formed of a material imageable from the second anatomical location.

18. The method of claim 16, further comprising expanding the anatomical wall at the first anatomical location to facilitate imaging thereof at the second anatomical location.

19. The method of claim 16, further comprising securing the locator device in the expanded configuration and separating the locator device from the delivery system used to deliver the locator device to the target site to deploy the locator device at the target site.

20. The method of claim 19, further comprising returning the locator device to the delivery configuration and removing the locator device from the target site.
